(19) United States
(12) Patent Application Publication
(10) Pub. No.: US 2017/0157102 A1
(43) Pub. Date: Jun. 8, 2017

(54) USE OF PERHEXILINE

(71) Applicants: CONSIGLIO NAZIONALE DELLE RICERCHE, Roma (IT); C.N.C.C.S. S.c.a.r.l. COLLEZIONE NAZIONALE DEI COMPOSTI CHIMICI E CENTRO SCREENING, Pomezia (IT); IRBM SCIENCE PARK S.p.A., Pomezia (IT)

(72) Inventors: Giovina RUBERTI, Monterotondo (IT); Cristiana LALLI, Monterotondo (IT); Alessandra GUIDI, Monterotondo (IT); Alberto BRESCIANI, Pomezia - Roma (IT); Nadia GENNARI, Pomezia - Roma (IT); Giacomo PAONESSA, Pomezia - Roma (IT); Emanuela NIZI, Pomezia - Roma (IT)

(21) Appl. No.: 15/325,920

(22) PCT Filed: Jul. 16, 2015
(86) PCT No.: PCT/EP2015/066264

§ 371 Date: Jan. 12, 2017

(30) Foreign Application Priority Data
Jul. 16, 2014 (IT) RM2014A000390

(51) Int. Cl. A61K 31/4458; A61K 31/4985

(52) U.S. Cl. CPC A61K 31/4458; A61K 31/4985

(57) ABSTRACT

The present invention relates to Perhexiline, or a pharmaceutically acceptable salt thereof, for use in the treatment of a pathology caused by trematodes.

### FIELD OF THE INVENTION

The present invention relates to the use of Perhexiline for the treatment of a pathology caused by a trematode or flukes, in particular schistosomiasis or bilharzia. Today, Praziquantel is the only widely available drug to treat schistosomiasis, the second most common parasitic disease in the world. The aggressive and repeated treatment campaigns to combat schistosomiasis raise increasing concern about the possible emergence of resistance. Moreover, the insensitivity of immature parasites represents the most serious problem in the clinical use of Praziquantel.

### BACKGROUND OF THE INVENTION

Trematodes are commonly referred to as flukes. This term refers to the flattened, rhomboidal shape of the worms. The flukes can be classified into two groups, on the basis of the system which they infect in the vertebrate host. Tissue flukes infect the bile ducts, lungs, or other biological tissues. This group includes the lung fluke, Paragonimus westermani, and the liver flukes, Clonorchis sinensis and Fasciola hepatica. Blood flukes inhabit the blood in some stages of their life cycle. Blood flukes include species of the genus Schistosoma. They may also be classified according to the environment in which they are found. For instance, pond flukes infect fish in ponds.

Human infections are most common in Asia, Africa, South America, or the Middle East. However, trematodes can be found anywhere where human waste is used as fertilizer. Schistosomiasis (also known as bilharzia, bilharziosis or snail fever) is an example of a parasitic disease caused by one of the species of trematodes (platyhelminth infection, or “flukes”), a parasitic worm of the genus Schistosoma. Other diseases caused by trematodes include Clonorchiasis, Paragonimiasis, Cercarial Dermatitis.

Schistosomiasis, one of the world's greatest human neglected tropical diseases, is caused by infection due mainly to Schistosoma mansoni, S. haematobium, or S. japonicum. Humans can become infected when their skin comes in contact with freshwater contaminated with the infectious larval stage of the parasite, known as cercariae. Among human parasitic diseases, schistosomiasis ranks second behind malaria in terms of socio-economic, public health importance and prevalence in the developing world, with more than 200 million people currently infected every year in 77 countries worldwide (85% in sub-Saharian Africa). The number of people treated for schistosomiasis rose from 12.4 million in 2006 to 33.5 million in 2010, in spite of treatment campaigns organized by the World Health Organization (WHO). It is estimated that 600 million people are at risk of infection and at least 280.000 deaths per year are associated with the severe consequences of infection, including fibrosis and calcification of the urinary tract, renal failure or bladder cancer (S. hematobium) and acute hepatitis, liver and intestine fibrosis, and portal hypertension (S. mansoni). To date no vaccine is available against schistosomiasis. After intensive research for the development of new chemotherapies in the middle of the last century, limited subsequent research efforts have reduced the therapeutic arsenal against this parasitic disease to one single drug: Praziquantel (PZQ). Since 1980s PZQ is the drug of choice for the treatment of schistosomiasis, because it is orally effective against all species of schistosomes with a single dose treatment. However, the in vivo efficacy of PZQ is dependent on the age of the infection, on the sex of the worms, and on their paired o unpaired status. During the earliest stages (from cercariae to the first few days after infection) the parasites are susceptible, followed by progressive insensitivity down to a minimum at around three to four weeks after infection (depending on the schistosome species). Schistosomes then gradually regain susceptibility until they are fully affected by the drug, around weeks 6-7 after infection. The ED₅₀ of PZQ against juvenile S. mansoni worms in mice (4 weeks after infection) was at least 30 times higher than that observed for adult worms (6 or 7 weeks) (Pica-Mattoccia et al., 2004). This can partially explain the low cure rates and rapid re-infection rates in endemic areas where patients are likely to be infected with juvenile and adult parasites concurrently (Dabo et al., 2000; N'Goran et al., 2003). The striking drug insensitivity of immature worms (between 1-5 weeks after infection) is actually the most serious problem in the clinical use of PZQ. For this reason re-treatments are often necessary. Multiple PZQ treatments and repeated rounds of mass treatments raise concerns about the development of resistance to PZQ. Remarkably, it is possible to induce resistance of S. mansoni and S. japonicum to PZQ in mice under laboratory conditions and resistance, reduced susceptibility or low cure rates to PZQ in the field isolates of S. mansoni has been sporadically reported (Fallon et al, 1996; Ismail et al, 1994; Gryseels, 2001; Cioli D, 2004; Melman S D et al, 2009). For the above reasons, the WHO has classified schistosomiasis as an illness for which new therapies are urgently needed (Gray D J, 2010).

Perhexiline (2-(2,2-dicyclohexylethyl) piperidine) (PHX) is a modulator of myocardial metabolism that is effective in the treatment of patients with refractory angina unsuitable for revascularization (Cole et al, 1990). More recently, it has also been shown to improve myocardial energetics and function in chronic cardiac failure (Lee et al, 2005) and symptomatic hypertrophic cardiomyopathy (Abozguia, et al. 2010). Historically, there have been difficulties in balancing the clinical effectiveness of PHX with significant toxicity, due to marked inter-individual variation in its pharmacokinetics, principally differences in elimination resulting from genetic polymorphisms of CYP2D6. These polymorphisms give rise to approximately 100-fold inter-individual differences in apparent oral clearance and plasma half-lives that range from 1-2 days in most subjects and up to 40 days in poor metabolizers. Severe adverse events, including hepatotoxicity and peripheral neuropathy, can be avoided by therapeutic drug monitoring to maintain plasma PHX concentration within a defined therapeutic range (0.15-0.6 mg 1⁻¹) (Horowitz, et al 1986). PHX is still believed to hold a critically important place in Australia and New Zealand for the treatment of patients with refractory angina or those who have contraindications to other standard anti-anginal therapies (Ashrafian et al. 2007; Lee et al. 2005). Remarkably, it has been also recently demonstrated in WO2014/036603 that the adverse effects observed upon administration of racemic perhexiline are unexpectedly associated with the (+) enantiomer and not the (−) enantiomer. PHX's main mode of action is ascribed to its inhibition of long-chain fatty acid oxidation by targeting of carnitine palmitoyltransferase (CPT) 1 and 2 (Kennedy J A et al., 2000). Biochemical assays showed also that PHX compound stimulates autophagy and inhibits mTORC1 signaling in cells maintained in nutrient-rich conditions (Balgi A D et al., PlosOne, 2009).

In a study described by Redman et al., Perhexiline was used to investigate the metabolism of sphingomyelin in schistosomes. In synthesis, treatment of adult parasites with the lysosomotrophic agents NH₄Cl, perhexiline and desipramine resulted in no decrease in the rate of BODIPY FL C₅-sphyngomyelin breakdown, suggesting that sphyngomyelin breakdown does not occur in the lysosome.

In order to search for clinically applicable drugs for schistosomiasis, the authors of the present invention screened 1280 FDA-approved drugs with a luminescent assay based on quantitation of the ATP, which signals the presence of metabolically active cells and organisms. Using schistosomula few hit compounds have been identified and among those Perhexiline (2-(2,2-dicyclohexylethyl)piperidine).

WO2013/182519 relates to pharmaceutical compositions comprising a lysosomotropic agent or agent modulating autophagy and a GSK-3 (glycogen synthase kinase 3) inhibitor, useful in the treatment of cancer, proliferative inflammatory diseases, degenerative diseases and infectious diseases including malaria, hepatitis A to C, African trypanosomiasis, cryptosporidiosis, Dengue fever, leishmaniasis, tuberculosis and schistosomiasis. The large list of lysosomotropic agents includes perhexiline. However there is no indication that perhexiline alone is effective to treat pathologies caused by a trematode, in particular as schistomicidal agent, preferably effective against juvenile and adult parasites.

Taylor C M et al. describe the efficacy of perhexiline in two nematode species, Haemonchus contortus and Onchocerca lienalis. There is no indication or evidence that the compound is active on trematodes too.

Nematodes and trematodes are very different parasites. Nematodes have a simple body form, often referred to as a “tube within a tube,” with a simple digestive system that extends from the mouth at one end to the anus at the other. Trematodes have flat, unsegmented bodies usually shaped like a leaf or an oval. Nematodes have two sexes and reproduce sexually. Except for members of the Schistosoma genus, trematodes are hermaphroditic, meaning they possess reproductive organs of both sexes. Their attachment mechanisms are also different. Nematodes attach to their hosts via liplike or toothlike plates that surround their mouth openings. Food is sucked into the body cavity by the working of muscles that surround the opening. In some species that prey on plants, the mouth cavity has been modified into a hollow spear that can penetrate the plant tissue and withdraw food. Trematodes attach to their hosts with two suckers, one anterior and one posterior. Nematodes can cause a number of serious diseases in humans including ascariasis, hookworm diseases, whipworm disease, trichinosis, pinworm infection and strongyloidiasis. These infections primarily affect the intestines of hosts and are most common in impoverished areas where sanitation standards are low. Trematodes can infect the skin, intestines, liver, blood, brain, lungs and other tissues of hosts, and symptoms can be severe and potentially life-threatening. Further, unlike trematodes, nematodes are major agricultural pests.

Despite the common terminology, the only shared biological features of many “helminthes” are their metazoan origins and the ability to infect mammals. Schistosomes are part of the platyhelminths that include the cestodes (tapeworms) and other trematodes (flukes or flatworms). The phyla Nematoda (roundworms) include hookworms, whipworms, and filarial parasites. The split that led to Platyhelminthes and Nematoda occurred over 1 billion years ago, long predating the split between vertebrates and invertebrates (B. Hausdorf, 2000). Therefore results obtained with nematodes cannot be extrapolated to trematodes.

### SUMMARY OF THE INVENTION

The present invention relates to the treatment of parasitic diseases, in particular of parasitic diseases caused by trematodes. Examples of parasitic diseases include malaria, toxoplasmosis, trypanosomiasis, leishmaniasis, schistosomiasis, Clonorchiasis, Paragonimiasis, Cercarial Dermatitis. In particular, the present invention relates to the treatment of schistosomiasis. As a matter of fact, within the present invention it has been surprisingly found that Perhexiline is effective against trematodes, in particular of the Schistosoma genus, more particularly including Schistosoma mansoni, Schistosoma haematobium and Schistosoma japonicum Perhexiline is active against Schistosoma mansoni.

Perhexiline (2-(2,2-dicyclohexylethyl)piperidine) (PHX) is then a highly promising treatment for pathologies caused by trematodes, in particular as anti-schistosomal compound to be used as an alternative or supplement to PZQ. Remarkably, PHX is active against larvae and both immature (1-5 weeks old) and adult S mansoni worms in vitro. The efficacy of PHX was also demonstrated in a murine model of S. mansoni. The use of PHX in the treatment of schistosomiasis can offer a solution to the major limitation that PZQ is not effective against juvenile parasites (4 weeks old). The use of PHX alone or in combination with PZQ can solve the problem of re-treatments. Moreover it can represent a valid alternative in case of more serious cases of emerging resistance to PZQ.

Importantly, PHX is currently used in chronic heart failure and refractory angina. Facing substantial obstacles to developing new therapies for neglected diseases, ‘repurpose’ drugs already approved for other conditions could speed the delivery of new therapies to people in need thereof. The invention relates to the use of Perhexiline for the treatment of schistosomiasis. The authors of the present invention have surprisingly found that Perhexiline acts as schistomicidal agent effective against juvenile and adult parasites.

In one aspect, the present invention provides the Perhexiline compound of formula (I):

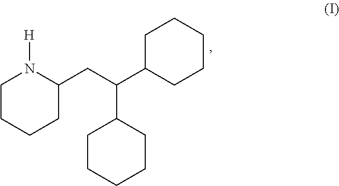 [SMILES: C1CCC(C(CC2CCCCN2)C2CCCCC2)CC1]

pharmaceutically acceptable salts or stereoisomers thereof for use in the treatment of a parasitic pathology or disease, in particular a pathology or disease caused by trematodes.

Preferably the compound is the (−)-enantiomer.

Preferably the pathology caused by trematodes is selected from the group consisting of: Schistosomiasis, Clonorchiasis, Paragonimiasis and Cercarial Dermatitis.

Still preferably the pathology caused by trematodes is Schistosomiasis.

In an embodiment the Schistosomiasis is caused by at least one of Schistosoma mansoni, Schistosoma haematobium, Schistosoma japonicum or a combination thereof.

In a preferred embodiment the trematodes are larvae, immature or juvenile trematodes or adult trematodes.

In a still preferred embodiment the pathology caused by trematodes is resistant to Praziquantel or oxamniquine or other anti-parasitic drug. Other anti-parasitic drug may include antimalarial agents (Atovaquone-proguanil, chloroquine, hydroxychloroquine, amodiaquine), metronidazole and tinidazole, nitazoxanide or paromomycin, ivermectin, Pyrantel Pamoate, Albendazole, mebendazole (Drug Therapy for Common Parasitic Infections Within the United States; Joel Thome et al; US Pharmacist 2012)

In one aspect, the present invention provides a pharmaceutical composition comprising the Perhexiline compound of formula (I) as defined above and at least one pharmaceutically acceptable excipient for use in the treatment of a parasitic pathology, preferably a pathology caused by trematodes.

Preferably the pharmaceutical composition further comprises at least another active compound.

Preferably the other active compound is Praziquantel or Oxamniquine or other anti-parasitic drug as defined above and known in the art. The combination may also comprises an anti-inflammatory agent such as glucocorticoids.

Preferably the other active compound is not a GSK-3 (glycogen synthase kinase 3) inhibitor.

In a preferred embodiment the dosage of Perhexiline ranges between 0.01 mg/kg/day to 100 mg/kg/day.

As used herein, the term “Perhexiline” refers to the chemical compound 2-(2,2-dicyclohexylethyl)piperidine, corresponding chemical formula (I):

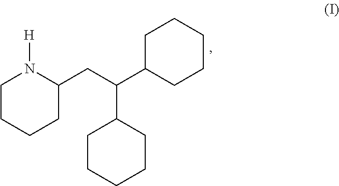 [SMILES: C1CCC(C(CC2CCCCN2)C2CCCCC2)CC1]

Included in the instant invention is the free base of Perhexiline as well as the pharmaceutically acceptable salts. The encompassed pharmaceutically acceptable salts include all the typical non-toxic pharmaceutically acceptable salts of the free form of the compound of formula (I). The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free form may differ from their respective salt forms somewhat in certain physical properties, such as solubility in polar solvents, but the salts are otherwise pharmaceutically equivalent to their respective free forms for purposes of the invention. Conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Preferred salts are the maleate salt, the hydrochloride salt or the lactate salt.

Perhexiline exists in two enantiomeric forms, and occurs as racemate and single enantiomers, the (+)-enantiomer and the (−)-enantiomer, all such stereoisomers being included in the present invention. As used herein, the term Perhexiline encompasses the racemate and the single enantiomers.

Perhexiline may be administered to a subject in a suitable form. In this regard the term “administering” includes administering perhexiline as a racemic mixture or single enantiomer, and/or administering a salt, prodrug or derivative of Perhexiline, that will form an effective amount of the active agent within the body of the subject. The term includes routes of administration that are systemic (e.g. via injection such as intravenous injection, orally in a tablet, pill, capsule, or other dosage form useful for systemic administration of pharmaceuticals). Methods of drug administration are generally known in the art.

The invention also provides pharmaceutical compositions comprising Perhexiline, alone or in combination with other anti-parasitic agents, and a pharmaceutically acceptable carrier.

When Perhexiline is administered into a human subject, the daily dosage regimen will normally be determined by the prescribing physician with the dosage generally varying according to the age, weight, sex and response of the individual patient, as well as the severity of the patient's symptoms. In one exemplary application, oral dosages of the present invention will range between about 0.01 mg per kg of body weight per day (mg/kg/day) to about 100 mg/kg/day, preferably 0.01 to 10 mg/kg/day, and most preferably 0.1 to 5.0 mg/kg/day.

Included within the scope of the present invention is Perhexiline in combination with known therapeutic schistosomiasis agents for simultaneous, separate or sequential administration and for treatment of polyparasitism that appears to be the rule, rather than the exception, both at the population level and among individuals residing in developing countries.

In an embodiment, Perhexiline may be used in combination with known agents useful for treating or preventing parasitic diseases, including malaria, toxoplasmosis, trypanosomiasis, Chagas disease, leishmaniasis, schistosomiasis, amebiasis, giardiasis, clonorchiasis, fasciolopsiasis, lymphatic filariasis, onchocerciasis, thricomoniasis and cestodiasis. Combinations of Perhexiline with other agents useful for treating or preventing parasitic disease are within the scope of the invention. A person of ordinary skill in the art would be able to discern which combinations of agents would be useful based on the particular characteristics of the drugs and the disease involved.

In particular, the present invention refers to a combination comprising Perhexiline and/or Praziquantel and/or Oxamniquine. The combination or pharmaceutical composition of the invention does not include a glycogen synthase 3-kinase (GSK-3) inhibitor.

In the present invention, the term “subject” refers to a human or any non-human animal (e.g., mouse, rat, rabbit, dog, cat, cattle, swine, sheep, horse or primate). In many embodiments, a subject is a human being. A human includes pre and post natal forms. In certain embodiments of the present invention the subject is an adult, an adolescent or an infant. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder. Also contemplated by the present invention are the administration of the pharmaceutical compositions and/or performance of the methods of treatment in-utero.

In the present invention, the genus Schistosoma is composed of over twenty species, infecting mammalian hosts. The genus has been divided into four groups—indicum, japonicum, haematobium and mansoni. Thirteen species are found in Africa. Twelve of these are divided into two groups—those with a lateral spine on the egg (mansoni group) and those with a terminal spine (haematobium group). The four mansoni group species are: S. edwardiense, S. hippotami, S. mansoni and S. rodhaini. The nine haematobium group species are: S. bovis, S. curassoni, S. intercalatum, S. guineensis, S. haematobium, S. kisumuensis, S. leiperi, S. margrebowiei and S. matthei. The indicum group has three species: S. indicum, S. nasale and S. spindale. This group appears to have evolved during the Pleistocene. All use pulmonate snails as hosts. The japonicum group has three species: S. japonicum, S. malayensis and S. mekongi. All the species are part of the invention.

In the present invention the term “larvae” means the free swimming stage infective for the definitive host, the term “immature or juvenile worms” means schistosomes aged up to 6 weeks post infection, the term “adult worms” means schistosomes aged from 6 weeks post infection.

The present invention will be described by means of non-limiting examples referring to the following figures:

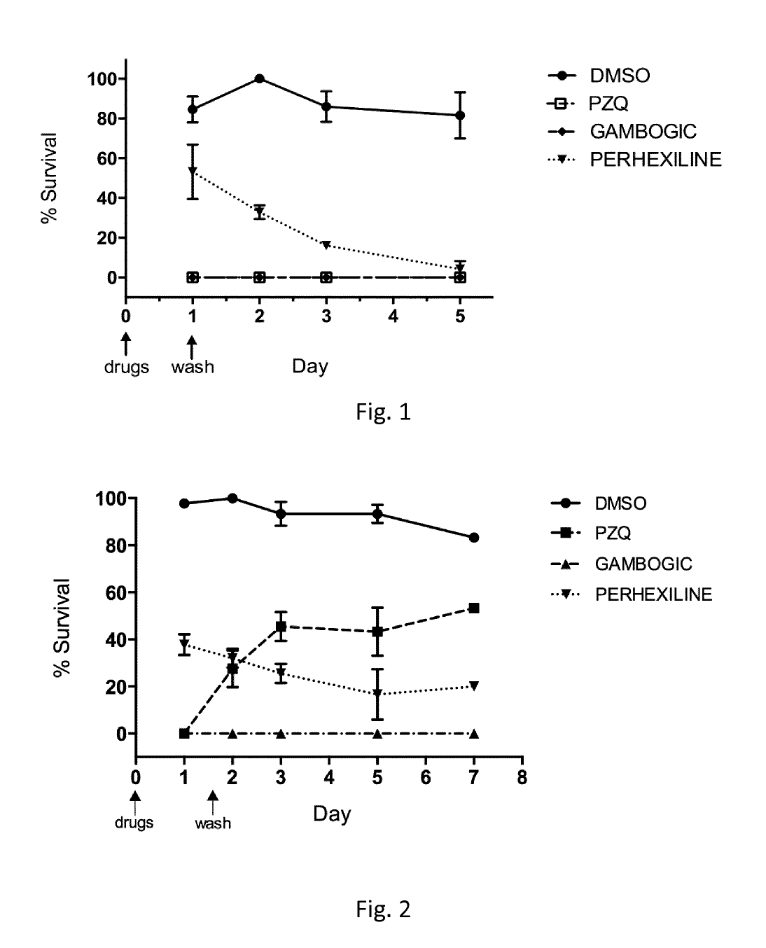

FIG. 1. Adult worms (8 weeks old, S. mansoni) in vitro survival following overnight treatment with 10 μM PHX, 10 μM Gambogic Acid, 10 μM Praziquantel (PZQ) or DMSO (vehicle). Parasites death was assessed by optical examination each day using the following criteria: reduction of motility, tegumental damages and darker appearance. Data shown are the means of results of at least three experiments.

FIG. 2. Juvenile worms (4 weeks old, S. mansoni) in vitro survival following overnight treatment with 10 μM PHX, 10 μM Gambogic Acid, 10 μM Praziquantel (PZQ) or DMSO (vehicle). Parasites death was assessed by optical examination each day using the following criteria: reduction of motility, tegumental damages and darker appearance. Data shown are the means of results of at least three experiments.

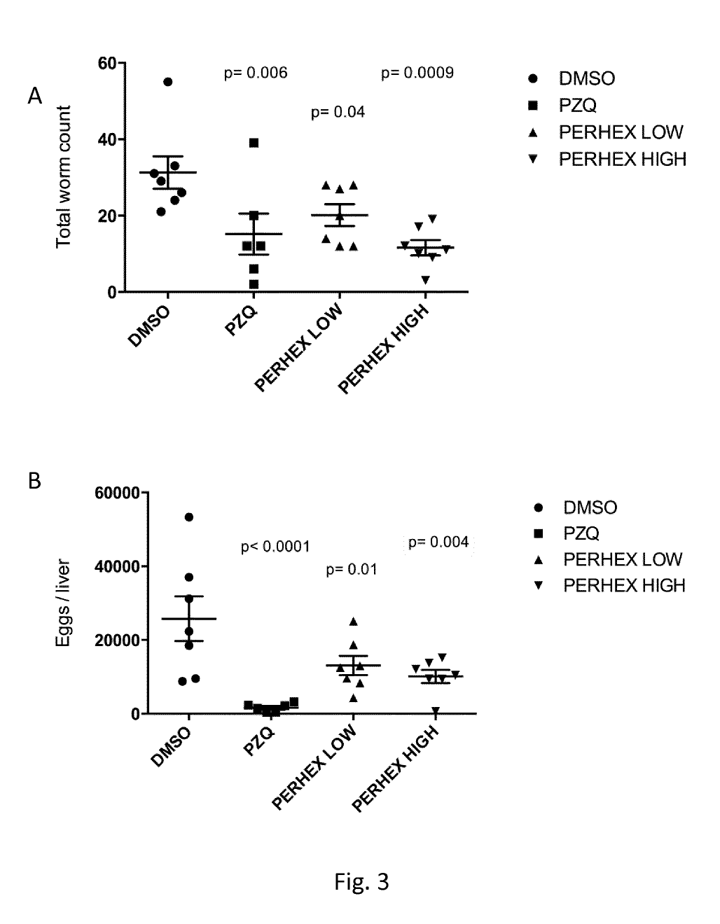

FIG. 3. Effect of PHX on total worm count (A) and egg burdens (B) in mice infected with S. mansoni. One-way analysis of variance (one-way Anova) was used to compare means of the samples. The p-values are indicated. PZQ (Praziquantel, oral administration, 500 mg/kg), PERHEX LOW (PHX, oral administration, 23 mg/kg), PERHEX HIGH (PHX, oral administration, 70 mg/kg).

### DETAILED DESCRIPTION OF THE INVENTION

### Material and Methods

Gambogic Acid, Perhexiline maleate salt, Praziquantel, Dimethyl sulphoxide (DMSO), Percoll (starting density 1.13 g/ml) and Foetal bovine serum (FBS) were purchased from Sigma-Aldrich. CellTiter-Glo reagent, used in the luminescent viability schistosomula assay, was purchased from Promega. BioWhittaker Dulbecco's Modified Eagle's Medium (DMEM) lacking phenol red but containing 4500 mg/l glucose (Lonza), supplemented with 1 mM Hepes (Lonza), 2mM L-glutamine (Lonza), 1×antibiotic-antimycotic reagent (Life Technologies) and 10% FBS was the completed tissue culture media for schistosomula. Juvenile and adult worms (S. mansoni) were cultured in BioWhittaker Dulbecco's Modified Eagle's Medium (DMEM) containing 4500 mg/l glucose (Lonza) supplement with 2 mM L-glutamine (Lonza), Penicilline 100 U/ml, Streptomycine 100 μg/ml (Lonza), Amphothericin B 0.5 μg/ml (Cambrex) and 10% heath inactivated FBS.

### Ethics Statement

All animals were subjected to experimental protocols as reviewed and approved by the Public Veterinary Health Department of the Italian Ministry of Health (Rome, Italy), according to the ethical and safety rules and guidelines for the use of animals in biomedical research provided by the relevant Italian laws and European Union's directives.

Maintenance of the S. mansoni Life Cycle

A Puerto-Rican strain of S. mansoni was maintained by passage through albino Biomphalaria glabrata, as the intermediate host, and ICR (CD-1) outbred female mice (Harlan Laboratories). The snails had been individually infected with 8-12 miracidiae per snail. Snails were kept in tanks with dechlorinated tap water in a humid room simulating a 12 hour day and night cycle.

First shedding of cercariae occurred from 4 weeks post infection. Approximately 100-200 snails (size of snails: 6-11 mm) were placed twice under a direct 2000 lux lamp for 60 min at 27° C. The cercarial suspension was collected and used for the preparation of schistosomula. Adult parasites were harvested by reversed perfusion of the hepatic portal system of infected mice previously euthanized with peritoneal (i.p.) injections of Tiletamina/Zolazepam (800 mg/kg)+Xylazina (100 mg/kg).

Animals and S. mansoni Infection

Female ICR (CD-1) outbred and C57BL/6, 4-7 weeks old mice (Harlan Laboratories) were housed under controlled conditions [(22±2)° C.; (65±15)% relative humidity; 12/12 h light/dark cycle]. The mice received standard food and water ad libitum. Female ICR (CD-1) outbred mice were infected transcutaneously with approximately 80 (for mixed infection) or 200 (single sex) S. mansoni cercariae for life cycle maintainance and in vitro worm assays. For in vivo experiments, C57BL/6 mice were infected with 140 cercariae and administered with selected compounds.

### Preparation of Schistosomula

Cercariae, shed from infected snails, were subsequently converted to schistosomula by mechanical transformation using an optimised version of the protocol of Brink et al., 1977 previously described (Protasio et al. 2013). Briefly, the cercarial suspension (approximately 50.000 cercariae) was placed in a glass 40 ml tube on ice for 60 minutes in order to reduce parasite motility. Tail detachment was obtained by shaking cercariae vigorously for approximately 30 seconds in a vortex mixer before passing these through a 22G syringe needle approximately 10-12 times. Next, the separation of heads/schistosomula and tails was obtained by placing the heads plus tails suspension on 4-5 ml of ice-cold 70% Percoll in tube and centrifugating (600×g) for 10 minutes at 4° C. Finally, the schistomula preparations were washed twice (with DMEM complete media lacking FBS) and microscope examination was used to assess the quantity and quality of purified schistosomula (less than 1% tails). Schistosomula were cultured at 37° C. in 6 wells tissue culture plates containing 3 ml schistosomula complete media in an atmosphere of 5% CO₂ for 24 hr before any further experimental manipulations proceeded. Negligible parasite death occurred in this media during the 24 hr culturing period. Following this, schistosomula were aliquoted into flat-bottom 384-well black-sided for compound assays.

### Screening of Compounds

A compound set of 1280 molecules comprising all the FDA, EMEA and other agencies approved drugs (Prestwick chemicals, Fr) was tested according to the following procedure.

### a) Compound Storage and Transfer to Assay Plates.

Compounds are stored as solution of 100% DMSO at −20 ° C. under inert atmosphere. Intermediate storage microplates, in the 384 well/plate format, are prepared on demand and stored in the same controlled environment as the stock solutions. Intermediate microplate stored compounds are transferred to assay plates by the acoustic droplet ejection technology (ATS-100, EDC Biosystems USA) which ensures a safe, contactless, pre-dilution free delivery. The test set was transferred to 384 well, black, tissue culture treated destination plate. The initial test was carried out at a single concentration of 10 μM.

### b) Assay Protocol.

A suspension of schistosomula, in DMEM added with 10% FBS, was transferred to each well of the compound containing assay plates in order to have 100 schistosomula per well in a final volume of 30 μL. The plate set was let to incubate with the compounds at 37° C./5% CO₂ for 24 hours. At the end of the incubation period each well was filled by and equal volume (30 μL) of CellTiterGlo (Promega Corp. P/N G7570) and let to incubate for 30 minutes at room temperature. The light based single emitted by the reaction is proportional to the ATP amount in the culture which ultimately reflects the mitochondrial function and thus the schistosomula viablity. The readout, luminescence based, was carried out by a CCD based detector (ViewLux, PerkinElmer USA). Each plate contains 16 DMSO treated samples as negative controls and 16 gambogic acid treated samples as positive controls.

### c) Active Compound Selection and Confirmation.

In order to select the best cytotoxic compounds, the effect of each molecule was resealed between 0% cytotoxicity (DMSO treated samples) and 100% cytotoxicity (gambogic acid treated samples). A threshold was set to 70% cytotoxicity that, together with other presumably cytotoxic compounds, surprisingly showed PHX as positive compound.

The latter molecule was purchased from the provider (Cas# 6621-47-2; Prestwick Chemicals, FR) as fresh lot and subjected to the UPLC-MS quality control. After having passed the QC, the new batch of PHX was tested in a dose response manner. To this aim, a serial dilution of the compound was carried out in DMSO in order to cover the concentration range between 50 μM and 20 nM. The transfer of the serially diluted compounds and the assay protocol were identical to those described in the previous paragraphs.

In vitro Studies with S. mansoni Juvenile (4 weeks old) and Adult (8 wEeks or Older) Worms

Seven-eight male worms or 4-5 worm pairs were recovered under aseptic conditions from infected mice by perfusion of mesenteric veins at 28 days (juvenile) or 56-days post infection (adult pairs) or 2-4 months post-infection (adult males). After washing, the worms were transferred into a 35 mm plates containing 3 mL of DMEM complete worms media and incubated at 37° C. in a humid atmosphere containing 5% CO₂ with drugs overnight. Next, the worms were washed 3 times with drug-free medium and were incubated for 5 days at 37° C./5% CO₂ and monitored daily under a stereo microscope for mobility, tegumental damage, viability and egg output (worm pairs). The experiment was repeated at least three times.

In vivo Studies with S. mansoni Infected Mice

C57B/L6 inbred female mice (5-6 weeks old) were divided in 4 groups of 7 animals each and treated with DMSO (control), PZQ or two dosages of PHX. All compounds were administered orally (vehicle is 2.5% Cremophor EL) 42 days post infection. The amount of PHX administered (23 mg/kg and 70 mg/kg) was guided by available information of therapeutic dose in use for angina treatment (Ashrafian H., 2007); PZQ was used at the standard dose (500 mg/Kg). Toxicity of compounds (e.g., death and behavioral changes) was assessed daily during and after treatment until the time of euthanasia. At 56 days post infection, mice were euthanized with an intraperitoneal injection of Tiletamina/Zolazepam (800 mg/kg)+Xylazina (100 mg/kg) and adult worms perfused as described above.

Compound efficacy in vivo, measured using a number of criteria, was compared to that of the anti-schistosomal drug, PZQ. The criteria include the numbers of male and female worms (total worm count) recovered by perfusion and hepatic egg burden (number of eggs in the liver). To recover eggs trapped in liver, whole livers from individual mice were excised, weighed and incubated in a 4% KOH solution over night at 37° C. Eggs were counted under a dissecting microscope as previously described (Cheever, A. W., 1968).

### Results

Efficacy of PHX on Schistosomula in vitro

In order to investigate the effect of PHX on S. mansoni larvae viability, newly transformed schistosomula were cultured in vitro for 24 h in presence of a range of drug concentrations (0.019-50μM). Parasites survival was assessed by using a CellTiterGlo luminescent assay and the CC₅₀ (calculated by fitting the four parameters equation) is shown in Table 1.

TABLE 1
Perhexiline and Gambogic Acid
CC₅₀ of newly transformed schistosomula

| Name | CC₅₀ (μM) |
|---|---|
| Perhexiline | 9.564; 12.391 |
| Gambogic Acid | 1.043; 2.138 |

CC50 values of two independent experiments

Gambogic Acid, previously described as a potent killer agent for schistosomes (Peak et al, 2010), was used as positive control and DMSO (drug vehicle) as negative control. Gambogic Acid, was confirmed to be effective in killing the schistosomula within the expected concentration range. Perhexiline was also shown to be effective in this validated model.

Efficacy of PHX on Different Stages of S. mansoni in vitro

Male adult worms (7-8 worms/sample) obtained from infected mice were cultured at 37° C., 5% CO₂ in DMEM complete medium (10% FBS) in presence of different concentrations of PHX (3-10 μM) and for variable time (12h to 5 days). For all experiments Gambogic Acid (1-10 μM) and PZQ (1-10 μM) were used as positive controls while DMSO was used as negative control. Overnight incubation with 10 μM PHX resulted in a statistically significant decreased viability of parasites after 5 days as shown in FIG. 1. Parasites death was assessed by optical examination each day using the following criteria: reduction of motility, tegumental damages and darker appearance.

PHX was also tested at the concentrations of 5 μM and 3 μM leading to a reduced viability (around 50%) in the first case and having no effect in the second one.

Juvenile worms (4 weeks old parasites) were also tested at the same conditions reported above. Following over night incubation of PHX (10 μM), viability of parasites was reduced by 80% after 5 days of observation (FIG. 2). Remarkably, PZQ (at the same concentration) treated worms started to recover after the wash at day 1 and PZQ treatment showed a 50% survival rate at 5 days. This is in accordance with previous report on PZQ showing a survival of approximately 80% of juvenile worms at 8 days (Livia-Pica et al, 2004).

In order to test the effect of PHX on egg production, adult worm pairs were treated overnight using the sub lethal dose of 5 μM. Total number of eggs laid by female parasites was determined 3 and 6 days after the treatment. To do so, the medium containing the eggs was harvested and briefly centrifuged. The pellet containing the eggs was re-suspended in 500 μl of saline solution and the egg number was determined by microscopy analysis. Results showed that PHX treatment led to a reduction in egg production of around 60% after both 3 and 6 days compared to the untreated control incubated with DSMO alone.

Efficacy of PHX in vivo in Mice Infected with S. mansoni

Based on the strong in vitro effects of PHX against larvae, juvenile (4 weeks old) and adult worms, the in vivo efficacy of the drug was tested in mice infected with S. mansoni. Based on the previous cardiovascular studies of the drug (Ashrafian H., 2007), two different concentrations of PHX were used: a low dose (23 mg/Kg) and a high dose (70 mg/Kg). The compound was administered orally as single dose in 2.5% Cremphor EL to mice with patent S. mansoni infection (56 days post infection). As positive control, PZQ was administered orally in a single dose at the standard concentration of 500 mg/Kg. Two weeks after the treatment, mice were sacrificed and the total worm count and egg burden (number of eggs in the liver) were determined (FIG. 3). A significant decrease in total worm counts was observed after treatment with both LOW and HIGH doses of PHX (35 and 63% respectively). For egg burden, a reduction of 50 and 60% was shown after treatment with LOW and HIGH doses of PHX, respectively.

As shown by the data reported herein, Perhexiline is a potent inhibitor of schistosoma worms with CC₅₀ in the low micromolar range. Worthy of specific note is the fact that the compound is active against larvae and both juvenile and adult worms, thus overcoming one of the major limitations of PZQ, the only drug currently in use for the treatment of schistosomiasis.

In addition, repurposing FDA or EMA-approved products has several practical advantages over novel compounds that are as yet unapproved for use in treating human diseases. With the tested dosages and formulation, approved products have not only demonstrated their pharmacological activity but have known toxicity profiles in humans and have well-studied pharmacokinetics and pharmacodynamics.

### REFERENCES

Abozguia K, Elliott P, McKenna W, Phan T T, Nallur-Shivu G, Ahmed I, Maher A R, Kaur K, Taylor J, Henning A, Ashrafian H, Watkins K, Frenneaux M. Metabolic modulator perhexiline corrects energy deficiency and improves exercise capacity in symptomatic hypertrophic cardiomyopathy. Circulation 2010; 122: 1562-1569.
Ashrafian H, Horowitz J. D., Frenneaux M. P. Perhexiline.Cardiovasc Drug Rev 2007, 25: 76-97
Cioli, D., S. S. Botros, K. W. Francklow, A. Mbaye and V. Southgate et al., 2004. Determination of ED50 values for praziquantel in praziquantel-resistant and susceptible Schistosoma mansoni isolates. Int. J. Parasitol., 34: 979-987
Cole P L, Beamer A D, McGowan N, Cantillon C O, Benfell K, Kelly R A, Hartley L H, Smith T W, Antman E M. Efficacy and safety of perhexiline maleate in refractory angina. A double-blind placebo-controlled clinical trial of a novel antianginal agent. Circulation 1990; 81: 1260-1270.
Balgi A D, Fonseca B D, Donohue E, Tsang T C F, Lajoie P, Proud C G, Nabi I R, Roberge M. Screen for chemical modulators of autophagy reveals novel therapeutic inhibitors of mTORC1 signaling. PlosOne 2009, Vol. 4, Issue 9, e7124.
Brink L H, McLaren D J, Smithers S R (1977) Schistosoma mansoni: a comparative study of artificially transformed schistosomula and schistosomula recovered after cercarial penetration of isolated skin. Parasitology 74: 73-86
Cheever, A. W. 1968. Conditions affecting accuracy of potassium hydroxide digestion techniques for counting Schistosoma mansoni eggs in tissues. Bull. World Health Organ. 39:328-331
Dabo, A.; Doucoure, B.; Koita, O.; Diallo, M.; Klinkert, M. Q.; Doumbia, S.: Soumbo, O. Reinfection with Schistosoma haematobium and mansoni despite repeated praziquantel office treatment I Niger, Mali. Medecine Tropicale: revue du corps de sante colonial (2000), 60(4), 351-355.
Fallon P G, Tao L F, Ismail M M, Bennett J L 1996. Schistosome resistance to praziquantel: fact or artifact? Parasitol Today 12: 316-320.
Gray D J, McManus D P, Li Y, Williams G M, Bergquist R, et al. 2010. Schistosomiasis elimination: lessons from the past guide the future. Lancet Infect Dis 10: 733-736.
Gryseels B, et al: Are poor responses to praziquantel for the treatment of Schistosoma mansoni infections in Senegal due to resistance? An overview of the evidence. Trop Med Int Health 2001; 6:864-873.
Hausdorf B., “Early evolution of the Bilateria,” Systematic Biology, vol. 49, no. 1, pp. 130-142, 2000.
Horowitz J D, Sia S T, Macdonald P S, Goble A J, Louis W J. Perhexiline maleate treatment for severe angina pectoris-correlations with pharmacokinetics. Int J Cardiol 1986; 13: 219-229.
Ismail M M, Taha S A, Farghaly A M, el-Azony A S 1994. Laboratory induced resistance to praziquantel in experimental schistosomiasis. J Egypt Soc Parasitol 24: 685-695.
Kennedy J A I, Kiosoglous A J, Murphy G A, Pelle M A, Horowitz J D. Effect of perhexiline and oxfenicine on myocardial function and metabolism during low-flow ischemia/reperfusion in the isolated rat heart. J Cardiovasc Pharmacol. 2000 December; 36(6):794-801.
Lee L., Campbell R., Scheuermann-Freestone M., Taylor R., Gunaruwan P., Williams L. et al. Metabolic modulation with perhexiline in chronic heart failure: a randomized, controlled trial of short-term use of a novel treatment. Circulation 2005, 112: 3280-3288
Melman S. D., et al. 2009. Reduced susceptibility to praziquantel among naturally occurring Kenyan isolates of Schistosoma mansoni. PLoS Negl. Trop. Dis. 3:e504.
N'Goran, E. K.; Gnaka, H. N.; Tanner, M.; Utzinger, J. Efficacy and side effects of two praziquantel treatments against Schistosoma haematobium infection, among schoolchildren from Cote d′Ivoire. Annals of Tropical Medicine & Parasitology (2003), 97(1), 37-51.
Peak E., I. W. Chalmers, K. F. Hoffmann Development and Validation of a Quantitative, High-Throughput, Fluorescent-Based Bioassay to Detect Schistosoma Viability. PLoS Negl Trop Dis. 2010
Pica-Mattoccia L, Cioli D. Sex- and stage-related sensitivity of Schistosoma mansoni to in vivo and in vitro praziquantel treatment. Int J Parasitol. 2004 Mar. 29; 34(4):527-33.
Protasio A V, David W. Dunne, and Matthew Berriman, Comparative Study of Transcriptome Profiles of Mechanical- and Skin-Transformed Schistosoma mansoni Schistosomula. Plos Negl Trop Dis 2013; 7(3):e2091
Redman et al. Molecular and Biochemical Parasitology 90, 1997, p 145-153
Taylor C M et al. PlosOne, 2013, Vol. 9, Issue 8, e1003505.

1-4. (canceled)

5. The method according to claim 14, wherein the pathology caused by trematodes is selected from the group consisting of: Schistosomiasis, Clonorchiasis, Paragonimiasis and Cercarial Dermatitis.

6. The method according to claim 5, wherein the pathology caused by trematodes is Schistosomiasis.

7. The method according to claim 6, wherein the Schistosomiasis is caused by at least one of Schistosoma mansoni, Schistosoma haematobium, Schistosoma japonicum or a combination thereof.

8. The method according to claim 14 wherein the trematodes are larvae, immature trematodes or adult trematodes.

9. The method according to claim 14, wherein the pathology caused by trematodes is resistant to Praziquantel or oxamniquine or other anti-parasitic drug.

10. (canceled)

11. The method according to claim 14 further comprising administering at least another active compound.

12. The method according to claim 11 wherein the other active compound is Praziquantel or Oxamniquine.

13. The method according to claim 14, wherein the dosage of Perhexiline ranges between 0.01 mg/kg/day to 100 mg/kg/day.

14. A method for the treatment of a pathology caused by trematodes comprising administering to a subject in need thereof a therapeutically effective amount of Perhexiline compound of formula (I): 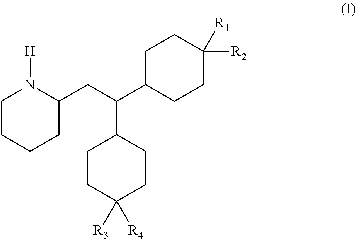
wherein R₁, R₂, R₃, R₄ are each independently —H or halogen, pharmaceutically acceptable salts or stereoisomers thereof.

15. The method according to claim 14, wherein R₁, R₂, R₃, R₄ are each independently —H or —F.

16. The method according to claim 14, wherein R₁, R₂, R₃, R₄ are —H.

17. The method according to claim 14, wherein said compound is the (−)-enantiomer.
